(19) United States
(12) Patent Application Publication
(10) Pub. No.: US 2014/0058065 A1
(43) Pub. Date: Feb. 27, 2014

(54) FAR-RED FLUORESCENT PROTEINS WITH IMPROVED DETECTABILITY BY RED EXCITATION LIGHT

(71) Applicant: The Board of Trustee of the Leland Stanford Junior University, (US)

(72) Inventors: Jun Chu, Mountain View, CA (US); Michael Z. Lin, Stanford, CA (US)

(73) Assignee: The Board of Trustees of the Leland Stanford Junior University, Palo Alto, CA (US)

(21) Appl. No.: 13/787,257

(22) Filed: Mar. 6, 2013

Related U.S. Application Data
(60) Provisional application No. 61/691,597, filed on Aug. 21, 2012.

(51) Int. Cl. C07K 14/435

(52) U.S. Cl. CPC C07K 14/43504
USPC 530/350

(57) ABSTRACT

Aspects of the present disclosure are directed towards far-red monomeric fluorescent proteins that have a high level of brightness such that the proteins are visible over the autofluorescence aspects of tissue. In certain embodiments, the fluorescent proteins are derived from Entacmeae quadricolor. The fluorescent proteins are implantable in the cell and can be excited by light of at least 600 nm.

### INCORPORATION-BY-REFERENCE OF MATERIAL SUBMITTED ELECTRONICALLY

Incorporated by reference in its entirety is a computer-readable nucleotide/amino acid sequence listing submitted concurrently herewith, and identified as follows: One 13,493 Byte ASCII (Text) file named “STFD314PA_ST25” and created on Mar. 6, 2013.

### BACKGROUND

Mammalian tissues are relatively transparent to red wavelengths of light within the “optical window” (600-1000 nm) because hemoglobin and myoglobin absorbances are relatively low. Engineering fluorescent proteins (FPs) to absorb beyond 600 nm (so called far-red fluorescent proteins) is desirable because the use of red light generates less tissue autofluorescence as compared to other wavelengths of light (such as blue light). Autofluorescence is the natural emission of light by biological structures (e.g., mitochondria, lysosomes, NAD(P)H, flavins, lipofuscin). Autofluorescence is used to distinguish the light originating from artificially added fluorescent markers. A large amount of autofluorescence of the tissue interferes with the ability to assess the response of a fluorescent protein in the tissue.

Monomeric fluorescent proteins (as opposed to oligomeric proteins) can be fused to other protein domains to create reporters of biochemical pathways or cellular states, such as kinase activity or cell cycle phases. Engineering monomeric fluorescent proteins having illumination wavelengths beyond the residual tail of hemoglobin absorbance (e.g., less affected by the autofluorescence of tissue) allows for commonly available 633-635 nm laser lines could then be used. The intrinsic peak brightness levels (the product of the peak extinction coefficient c and quantum yield φ) of some far-red FPs can be correlated with their redness. Unless otherwise specifically noted, use of the term “brightness” refers to the intrinsic brightness of the protein rather than the perceived brightness of the protein. The “perceived brightness” of a fluorescent protein refers to the level of brightness observed by a collector or analyst. This “perceived brightness” differs based on the quality of the collector or analyst, e.g., the optical properties of the imaging setup (illumination wavelength and intensity, spectra of filters and dichroic mirrors), and camera or human eye sensitivity to the emission spectrum. A factor in the perceived brightness of a fluorescent protein is the intrinsic brightness of the protein. The intrinsic brightness of a fluorescent protein is determined by its maturation speed and efficiency, extinction coefficient, quantum yield and, in longer experiments, photostability. The quantum yield (φ) of a fluorescent protein is a variable that displays the efficiency of the protein. Quantum yield is calculated by determining a ratio of the number of photons emitted by a fluorescent protein, versus the number of photons absorbed. The number of photons emitted relates to the visible cognizability (“glow”) of the fluorescent protein as a result of the proteins excitation. The photons absorbed relate to the wavelength of light utilized in the excitation of the fluorescent protein. A fluorescent protein having a quantum yield of 1 (100%), e.g., 100% efficient, would emit each photon that it absorbs. This level of quantum yield is not possible due to the laws of physics; however, a fluorescent protein having a quantum yield greater than 0.10 is considered to have a “high” brightness.

### SUMMARY

Aspects of the present disclosure are directed toward red-shifting of FP absorbance wavelengths beyond 600 nm while maintaining brightness, or improving brightness while maintaining redness. Certain embodiments relate to non-invasive imaging of cellular changes in vivo using any red-absorbing FP.

Various aspects of the present disclosure are directed toward fluorescent monomeric proteins derived from Entacmeae quadricolor. The fluorescent monomeric proteins, consistent with various aspects of the present disclosure, have an excitation peak of above 590 nm. At the excitation peak, the fluorescent monomeric proteins have a brightness of at least 20 mM⁻¹ cm⁻¹.

Various aspects of the present disclosure also include providing a fluorescent monomeric protein to a cell. An excitation light of at least 600 nm is then used to active the fluorescent monomeric protein.

The above discussion is not intended to describe each embodiment or every implementation. The figures and following description also exemplify various embodiments.

### BRIEF DESCRIPTION OF THE FIGURES

Various example embodiments may be more completely understood in consideration of the following detailed description in connection with the accompanying drawings, and those in the Appendices as were filed as part of the underlying provisional application.

FIG. 1 shows an example far-red monomeric fluorescent protein structure, consistent with various embodiments of the present disclosure;

FIG. 2 shows an example embodiment of an amino acid side chain with a length that is sufficient to donate a hydrogen bond a far-red monomeric fluorescent protein, consistent with aspects of the present disclosure;

FIG. 3 shows another example embodiment of an amino acid side chain with a length that is sufficient to donate a hydrogen bond a far-red monomeric fluorescent protein, consistent with aspects of the present disclosure;

FIG. 4 shows spectral characteristics of far-red monomeric fluorescent proteins, consistent with various embodiments of the present disclosure,

FIG. 4A shows an example absorbance spectra of oxygenated hemoglobin (oxyHb), deoxygenated hemoglobin (deoxyHb), and monomeric far-red FPs, consistent with various aspects of the present disclosure,

FIG. 4B shows an example normalized excitation (left) and emission (right) spectra of monomeric far-red FPs, consistent with various aspects of the present disclosure,

FIG. 4C shows an example) fluorescence emission intensities of far-red FPs when excited at 635 nm, consistent with various aspects of the present disclosure;

FIG. 5 shows an example normalized excitation and emission spectrum of mCardinal2 and other far-red FPs, consistent with various aspects of the present disclosure;

FIG. 6 shows an example characterization of far-red FPs, consistent with various aspects of the present disclosure,

FIG. 6A shows example mutation kinetics at 37° C. of far-red FPs, consistent with various aspects of the present disclosure,

FIG. 6B shows example green and red emissions detected of far-red FPs upon excitation with 460 nm light, consistent with various aspects of the present disclosure,

FIG. 6C shows example photobleaching kinetics of far-red FPs under arc lamp illumination with a 615/620 nm excitation filter, consistent with various aspects of the present disclosure,

FIG. 6D shows example pH dependence of fluorescence of far-red FPs, consistent with various aspects of the present disclosure;

FIG. 7 shows comparison of far-red monomeric fluorescent proteins, consistent with various embodiments of the present disclosure, for deep-tissue imaging,

FIG.7A shows equal amounts (8 μg) of purified far-red FPs, consistent with various aspects of the present disclosure, injected subcutaneously into ventral locations in BALB/c nude mice,

FIG. 7B shows quantization of fluorescence of far-red FPs, consistent with various aspects of the present disclosure, relative to mNeptune1 upon excitation with a 585-620 nm filter,

FIG. 7C shows example far-red FPs fluorescence, consistent with various aspects of the present disclosure, relative to mNeptune1 upon excitation with a 620-650 nm filter,

FIG. 7D show an example measurement of equal amounts (23 μg) of purified far-red FPs placed inside of a phantom mouse in a bore located 7 2 mm deep from the mouse surface, consistent with various aspects of the present disclosure,

FIG. 7E shows an example measurement of far-red FP contrast over the background (mean±SEM, n=3) upon excitation with a 585-620 nm filter, consistent with various aspects of the present disclosure,

FIG. 7F shows an example measurement of far-red FP contrast over the background (mean±SEM, n=3) upon excitation with a 620-650 nm filter, consistent with various aspects of the present disclosure;

FIG. 8 shows non-invasive longitudinal visualization of muscle regeneration, consistent with various aspects of the present disclosure,

FIG. 8A shows example representative fluorescence images of tibialis anterior muscles injected with 1 million myoblasts expressing mCardinal1, consistent with various aspects of the present disclosure,

FIG. 8B shows example acquired images from 3, 7, and 14 days post-injection (dpi), consistent with various aspects of the present disclosure,

FIG. 8C shows a magnified view of a muscle at 7 dpi, consistent with various aspects of the present disclosure,

FIG. 8D shows an example fluorescence signal from tibialis anterior muscles injected with 1000 satellite cells expressing mCardinal1, consistent with various aspects of the present disclosure,

FIG. 8E shows an example magnified view of muscle at 44 dpi injected with satellite cells, consist with various aspects of the present disclosure;

FIG. 9 shows an example comparison of mCardinal with mNeptune1 combined with iRFP in myoblasts, consistent with various aspects of the present disclosure,

FIG. 9A shows representative fluorescence images of tibialis anterior (TA) muscles injected with 1 million myoblasts expressing mNeptune1 or mCardinal and iRFP separated with P2A peptide,

FIG. 9B shows an example contrast of mNeptune1 (combined with iRFP) versus mCardinal (combined with iRFP) over background; and

FIG. 10 shows an example comparison of mCardinal with the brightest GFP, Clover, in muscle stem cells, consistent with various aspects of the present disclosure.

While the disclosure is amenable to various modifications and alternative forms, examples thereof have been shown by way of example in the drawings and will be described in detail. It should be understood, however, that the intention is not to limit the disclosure to the particular embodiments shown and/or described. On the contrary, the intention is to cover all modifications, equivalents, and alternatives falling within the spirit and scope of the disclosure.

### DETAILED DESCRIPTION

Aspects of the present disclosure are directed towards far-red monomeric fluorescent proteins that have a high level of brightness such that the proteins are visible over the autofluorescence aspects of tissue. In certain embodiments, the fluorescent proteins are derived from Entacmeae quadricolor (e.g., wild type eqFP578 protein from Entacmeae quadricolor). It is recognized that Entacmeae quadricolor has an excitation level in the far-red wavelength of light.

In various embodiments, the sequence derived from Entacmeae quadricolor can be altered to produce a protein having an excitation peak above 590 nm, and a brightness at that peak of at least 20. In this manner, the fluorescent proteins can also be developed with relatively high excitability within the range of available 633-635 nm laser lines. The fluorescent proteins can also be designed with visibility above the autofluorescence of the tissues in which the fluorescent proteins are inserted. The brightness achieved by far-red monomeric fluorescent proteins, consistent with certain embodiments discussed herein, can be further characterized in that the brightness of the protein when excited by a 635 nanometer light (e.g., a readily available laser light frequency) is at least 2. Further, at an excitation wavelength of 635 nm, certain fluorescent proteins can be engineered with a molar absorption coefficient that is at least 12 mM⁻¹ cm⁻¹.

Certain embodiments of the far-red monomeric fluorescent proteins of the present disclosure obtain the enhanced levels of brightness due to a mutation to a hydrophobic core of the protein, and at least one position in a beta barrel wall of the protein. Further, various aspects of the present disclosure are directed toward fluorescent monomeric protein having mutations to mNeptune1 at one or more of mutation locations and providing an excitation peak of above 590 nm.

Aspects of the present disclosure are directed toward far-red monomeric fluorescent proteins. Certain embodiments of the far-red monomeric fluorescent proteins of the present disclosure include a mutagenesis of a hydrophobic core of a monomeric autocatalytic fluorescent protein. Mutagenesis of the hydrophobic of the far-red monomeric fluorescent proteins was found to improve the maturation and/or the fluorescence (brightness) of the protein. Without being limited by theory, it is believed that these features are a result of packing of amino acid residues of the protein sequence that surround a chromophore utilized with the protein. The chromophore is the portion of the far-red monomeric fluorescent proteins that gives off color when excited by visible light. In order to verify this mutation of a hydrophobic core, structure-guided site-directed mutagenesis can be performed, and the varying maturation and brightness of the chromophore can then be observed.

Additionally, other embodiments of far-red monomeric fluorescent proteins of the present disclosure include a mutation in a cross-dimer interface of a monomeric autocatalytic fluorescent protein. Without being limited by theory, mutation of the cross-dimer interface is believed to remove hydrophobicity of the far-red monomeric fluorescent protein, which can be energetically (e.g., excitability) unfavorable due to for folding of the protein. As a specific example, mNeptune2 is a far-red monomeric fluorescent protein of the present disclosure having a mutation in the cross-dimer interface. mNeptune2 is a far-red monomeric fluorescent protein having a relatively high brightness.

In certain embodiments of far-red monomeric fluorescent proteins of the present disclosure, a portion of a chromphore of the far-red monomeric fluorescent protein receives a hydrogen bond from a water molecule. This hydrogen bond stabilizes an excited state of the far-red monomeric fluorescent protein in providing a greater electron density, relative to the ground state of the protein, of a carbonyl oxygen of the protein's chromophore.

Additionally, certain ones of far-red monomeric fluorescent proteins of the present disclosure include a genetically encoded hydrogen bond at a portion of the chromphore. Providing a genetically encoded hydrogen reduces excited state vibrations (which leads to non-radioactive decay) of the far-red monomeric fluorescent protein, and red-shifting of the protein by increasing the strength of the hydrogen bond interaction.

Certain embodiments of far-red monomeric fluorescent proteins of the present disclosure have saturation mutagenesis of at least one amino acid position in the beta wall of the amino acid sequence of the protein. Position of the mutagenesis occurs at a position facing the carbonyl oxygen portion of the chromophore of the far-red monomeric fluorescent protein. The side chain of the amino mutated amino acid in the beta wall has length sufficient to donate a hydrogen bond to the carbonyl oxygen of the far-red monomeric fluorescent protein's chromophore. In order to verify these characteristics of the far-red monomeric fluorescent proteins, structural modeling can be performed. As a result of verification techniques, a number of far-red monomeric fluorescent proteins having mutations in the beta wall facing the carbonyl oxygen portion of the chromophore have been developed. For instance, as a specific example of far-red monomeric fluorescent protein having these type of mutations, mNeptune2 was developed, consistent with various embodiments of the present disclosure. mNeptune2 includes three mutations in the amino acid sequence of the beta wall, and achieves a high level of brightness when excited by a laser wavelength that is approximately equal to 635 nm. mNeptune2 has an excitation peak greater than 590 nm. As another example of a protein falling within the instantly described embodiment, or mCardinal1 has an excitation peak greater than 590 nm, and has a high level of brightness at the excitation peak.

Each of the aspects described are not limiting examples, and can be utilized alone or in combination with other aspects in the far-red monomeric fluorescent proteins of the present disclosure.

The following discussion provides details of experimental embodiments. Although the experimental embodiments provide examples and details regarding various parameters and results, these aspects are not necessarily limiting to the various other embodiments of the present disclosure.

Aspects of the present disclosure are directed toward a bright monomeric autocatalytic fluorescent protein (mNeptune) that is a derivative of the eqFP578 protein from Entacmeae quadricolor. Experimentally, mNeptune has been found to have a peak excitation of 600 nm at the left boundary of the optical window. mNeptune can have a large Stokes' shift of 50 nm and high brightness 68 =67 mM⁻¹ cm⁻¹, φ=0.20) compared to some fluorescent proteins excitable beyond 600 nm. mNeptune, consistent with certain embodiments of the present disclosure, is excitable by light beyond 620 nm while having a high brightness in living animals (as compared to some monomeric and oligomeric autocatalytic fluorescent proteins). For example, experimental fluorescent tests of mNeptune placed in a high vascularized liver tissue (which is deeply situated in a patient) have shown detection of mNeptune with 30-fold contrast over the tissue background, which is greater than the 13-fold contrast of IFP1.4 (which utilizes more red-shifted excitation wavelengths).

Certain aspects of the present disclosure are directed towards a structure-guided site directed mutagenesis of the hydrophobic core of mNeptune which optimizes packing of residues surrounding the chromophore. This optimization can influence chromophore maturation and brightness. Embodiments of the present disclosure are also directed toward mutation of Ile-171 in the cross-dimer interface of mNeptune. This mutation can remove hydrophobicity that may be energetically unfavorable for monomer folding.

Aspects of the present disclosure are also directed toward fluorescent protein (mNeptune2) having internal mutations at A104V and I121L, with an external mutation I171H. mNeptune2 was observed to have an excitation and emission spectra similar to that of mNeptune1, but with a greater level of brightness. For instance, mNeptune2 was observed to have a brightness 59% greater (ε=89 mM⁻¹ cm⁻¹, φ=0.24) than mNeptunel.

In certain embodiments of the mNeptune1 crystal structure (and experimentally in mNeptune2), a water molecule donates a hydrogen bond to the carbonyl oxygen in the acylimine portion of the chromophore. This can have a bathochromic effect by preferentially stabilizing the excited state, in which electron density over the carbonyl oxygen is believed to be increased relative to the ground state. Aspects of the present disclosure are directed toward fluorescent proteins having a genetically encoded hydrogen bond donor substituted for the water molecule. This substation can result in improved brightness by reducing excited state vibrations that could lead to non-radiative decay, or cause further red-shifting by increasing the strength of the hydrogen bond interaction.

Aspects of the present disclosure are directed toward saturation mutagenesis of fluorescent proteins in the beta wall facing the carbonyl oxide.

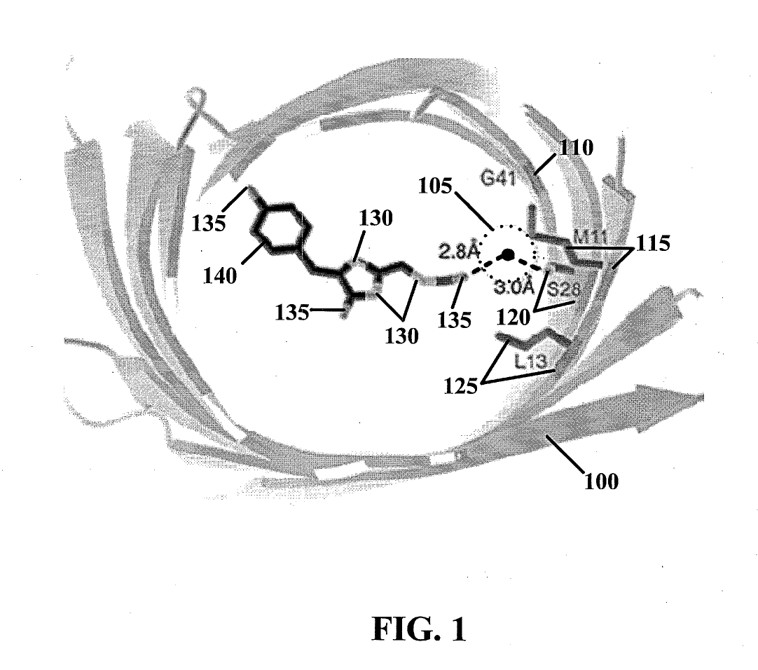

FIG. 1 shows an example beta wall structure 100 of the mNeptune fluorescent proteins, consistent with the present disclosure. The beta wall structure 100 displays a water molecule 105 which is hydrogen-bonded to an acylimine oxygen of the chromophore. In the center of the beta wall structure 100, the chromophore structure is depicted as the stick structure with carbon structures 140, nitrogen 130, and oxygen 135. FIG. 1 also shows positions for introduction of side chains (e.g., the mutation positions mentioned above with reference to mNeptune2, mNeptune2.5, and mCardinal1) : Met-11 (115), Leu-13 (125), Ser-28 (120) and Gly-41 (110). These side chains directly hydrogen bond with the acylimine oxygen.

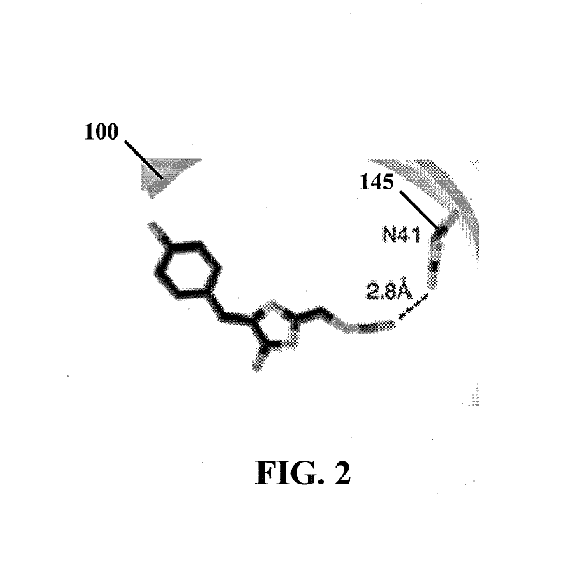

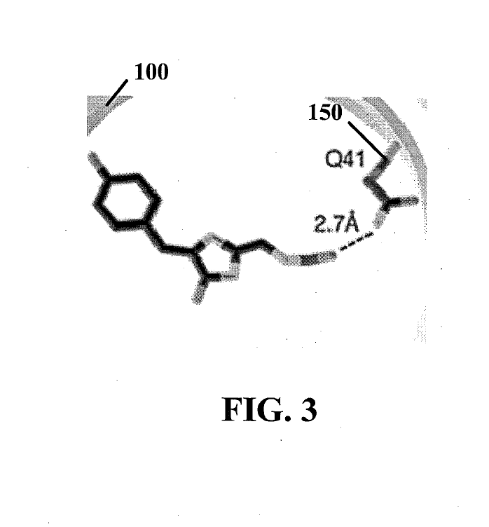

FIG. 2 and FIG. 3 show Asn (145) and Gln (150) at position 41 in an example beta wall structure 100 of fluorescence proteins consistent with the present disclosure. Also shown therein is the distance (e.g., 2.8 and 2.7 Å) between the amide nitrogen and the carbonyl oxygen atoms of the Asn (145) or Gln (150) bonded to the chromophore.

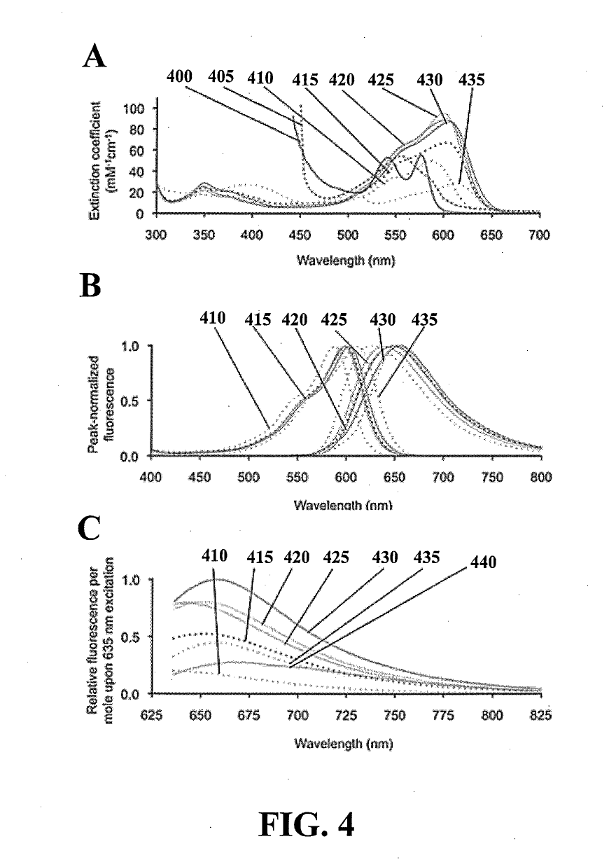

FIG. 4 shows spectral characteristics of far-red monomeric fluorescent proteins, consistent with various embodiments of the present disclosure. FIG. 4A shows a comparison of the absorbance spectra of oxygenated hemoglobin (oxyHb) 400, deoxygenated hemoglobin (deoxyHb) 405, and monomeric far-red FPs, consistent with various aspects of the present disclosure. The monomeric far-red FPs shown in FIG. 4A are mKate2 410, mNeptune1 415, mNeptune2 420, mNeptune2.5 425, mCardinal 430, and TagRFP657 435. FIG. 4B shows an example normalized excitation (left) and emission (right) spectra of monomeric far-red FPs, consistent with various aspects of the present disclosure. FIG. 4C shows example fluorescence emission intensities of far-red FPs when excited at 635 nm, consistent with various aspects of the present disclosure. Also shown in FIG. 4C is the far-red FP eqFp670 440. FIG. 4C shows an accurate reflection of the number of photons emitted at each wavelength upon excitation at 635 nm, for each FP (the area under the emission spectrum beyond 635 nm was normalized to the product of extinction coefficient at 635 nm and the quantum yield beyond 635 nm).

Aspects of the present disclosure are directed toward fluorescent proteins having mutagenized positions interacting with amino acids 28 and 41 and positions interacting with the phenolate group of the chromophore. Certain embodiments of fluorescent proteins having a mutation of the mNeptune2 sequence at M11T, S28H, and G41N. A far-red monomeric fluorescent protein having these specific mutations, called mNeptune2.5, has been observed to have a brightness (ε=95 mM⁻¹ cm⁻¹, φ=0.28) that is 18% brighter than mNeptune2, and brightness that is 92% brighter than mNeptunel. These experimental results found with respect to mNeptune2.5 also were found to have an excitation and emission spectra only slightly blue-shifted (peak ex/em, 599/643 nm).

Other embodiments of the present disclosure are directed towards fluorescent proteins having mutations of the mNeptune2 sequence at S28T, G41Q, and S143T have been preliminarily analyzed, and found to have a measured brightness (ε=87 mM⁻¹ cm⁻¹, φ=0.19) that is 23% brighter than mNeptunel, and has additionally red-shifted excitation and emission spectra (peak ex/em, 604/659 nm). Due to its monomeric character and its cardinal-red emission hue, fluorescent proteins having these characteristics are referred to as mCardinal1.

Additional embodiments of the present disclosure are directed towards fluorescent proteins having mutations of the mNeptune2 sequence at one or more of S28T, G41Q, S143T, N71K, T73P, Q74K, and V218E. Due to its monomeric character and its cardinal-red emission hue, fluorescent proteins having these characteristics are referred to as mCardinal2.

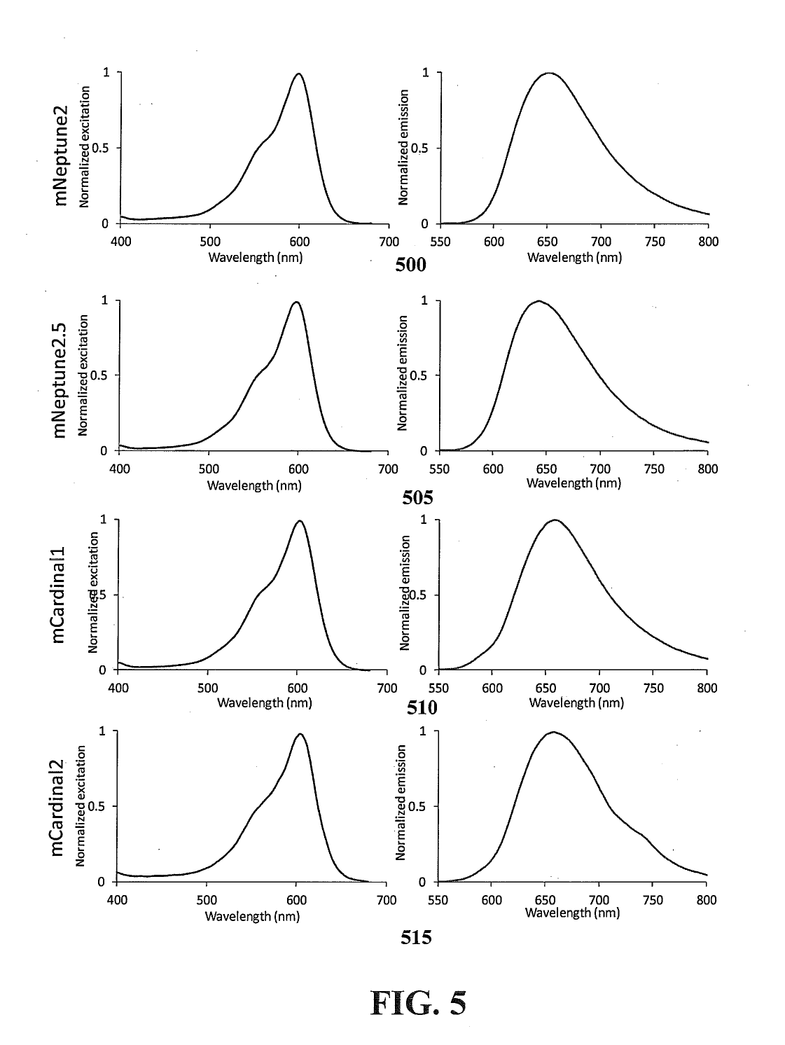

FIG. 5 shows an example normalized excitation and emission spectrum of mCardinal2 and other far-red FPs, consistent with various aspects of the present disclosure. The far-red FPs shown in FIG. 5 include mCardinal2 500, along with example normalized excitation and emission spectrums of mNeptune2 505, mNeptune2.5 510, and mCardinal1 515. In the table below, the example proteins discussed above are shown with corresponding mutations:

TABLE 1
Example Proteins and Mutations Consistent with Various Aspects of
the Present Disclosure

| | | | Maturation and | |
|---|---|---|---|---|
| | Red-Shift | Brightness | Folding | Monomericity |
| mNeptune2 | None | A104V and | A104V and I121L | I171H |
| | | I121L | | |
| mNeptune2.5 | S28H and | Above | Above + M11T | Above |
| | G41N | | | |
| mCardinal1 | S28T and | Above + | Above + S143T | Above |
| | G41Q | S143T | | |
| mCardinal2 | Above | Above | Above + N71K, | Above + |
| | | | T73P, and Q74K | V218E |

The specific mutations described above, and in the sequences listed below, are provided by way of example and given on a provisional basis. Additional or fewer mutations can be present in the protein sequences without departing from the embodiments described herein. Certain embodiments of the present disclosure are directed towards fluorescent proteins having S28H and G41N mutations, which have high fluorescence in bacteria. Other embodiments are directed towards fluorescent proteins having mutations at S28T and G41 Q, and which appear red-shifted in absorbance. Asn or Gln side-chain at position 41 can have a suitable length for its amide nitrogen to donate a hydrogen bond to the carbonyl oxygen of the chromophore. mNeptune2 and mCardinal1 have been found to be excitable at the laser wavelength of 635 nm (extinction coefficients 12 and 18 mM⁻¹ cm⁻¹, respectively) and can have at least 2-fold higher quantum yields above 635 nm than TagRFP657 and eqFP670 (two other autofluorescent FPs with similar absorptivity at 635 nm). Experiment results have found that the fluorescence emission from mNeptune2 and mCardinal1 can be greater than that of TagRFP657 and eqFP670 when excited at 635 nm, even at infrared wavelengths beyond 700 nm. Blue transmission of mCardinal1 is due to efficient absorbance of green and red light. At the excitation wavelengths (400-500 nm), the extinction coefficients of the fluorescent proteins are similar except for TagRFP657 (a), so emission intensity is proportional to quantum yield. TagRFP657 is actually more efficiently excited at 400-500 nm than the other fluorescent proteins.

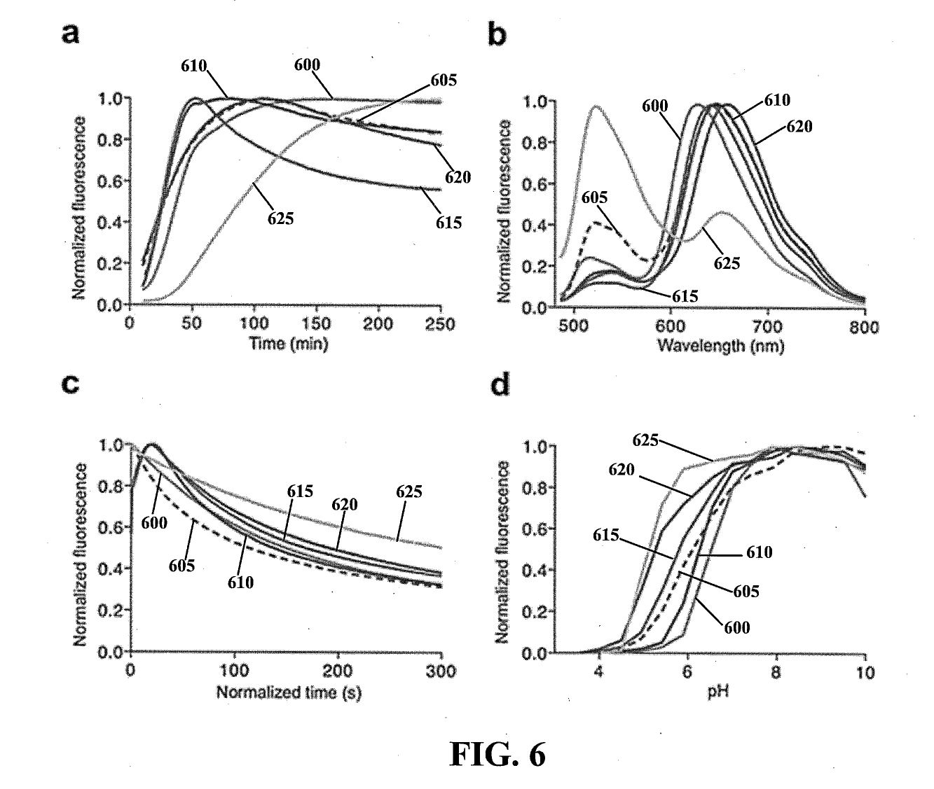

Certain aspects of the present disclosure include aspects directed toward fluorescent proteins, such as mNeptune2, mNeptune2.5, and mCardinal1, having far-red chromophore maturation of speed and completeness, residual green fluorescence, pH stability, and photostability. Compared to mNeptune1, various embodiments of fluorescent proteins can exhibit equal or faster red chromophore maturation at 37° C. Certain ones of fluorescent proteins of the present disclosure exhibit improved maturation efficiency (fraction of total protein forming chromophores). Table 2 compares experimental findings of proteins, consistent with the present disclosure, to previously developed proteins. Additionally, experimental results of this chromophore maturation are shown in FIG. 6.

TABLE 2
Characteristics of Monomeric Far Red FP's Consistent with the Present Disclosure

| | | | | | | mNeptune | |
|---|---|---|---|---|---|---|---|
| | mKate2 | TagRFP657 | eqFP670 | mNeptune 1 | mNeptune 2 | 2.5 | mCardinal |
| Excitation peak (nm) | 588 | 611 | 605 | 600 | 599 | 600 | 604 |
| Emission peak (nm) | 633 | 657 | 670 | 650^{g} | 651 | 643 | 659 |
| ε at peak (mM⁻¹cm⁻¹) | 50 | 29 | 70 | 67 | 89 | 95 | 87 |
| Φ | 0.40 | 0.095 | 0.06 | 0.20 | 0.24 | 0.28 | 0.19 |
| Brightness excited at peak | 20 | 2.9 | 4.2 | 13 | 21 | 27 | 17 |
| ε at 635 nm (mM⁻¹cm⁻¹) | 1.4 | 13 | 16 | 9.6 | 12 | 11 | 18 |
| Φ > 635 nm (mM⁻¹cm⁻¹) | 0.25 | 0.086 | 0.055 | 0.16 | 0.19 | 0.20 | 0.16 |
| Brightness excited at 635 nm | 0.35 | 1.1 | 0.88 | 1.5 | 2.2 | 2.2 | 2.8 |
| Maturation half-time (min) | 38 | 88 | ND | 28 | 27 | 26 | 27 |
| Maturation efficiency | 0.52 | 0.66 | ND | 0.48 | 0.53 | 0.52 | 0.60 |
| Photostability | 31 | 153 | ND | 100 | 76 | 127 | 218 |
| pKa | 6.5 | 5.1 | 4.5 | 5.4 | 6.3 | 5.8 | 5.3 |
| quaternary structure | m | m | d | m | m | m | m |
| (m = monomer, d = dimer) | | | | | | | |

In Table 2, the example excitation peak is represented by λex, and the example emission peak is represented by λem. Additionally, the quantum yield (φ) is calculated as the product of the extinction coefficient ε at λex and has units of mM⁻¹ cm⁻¹. Further, the brightness is calculated as the product c of at 635 nm with an emission fraction above 650 nm, and φ in units of mM⁻¹ cm⁻¹. The maturation half-time in Table 2 is an example calculated time for fluorescence to obtain half-maximal value after exposure to oxygen. Further, the maturation efficiency is calculated based on a functional chromophore concentration divided by total protein concentration. Additionally, the photostability calculated in Table 2 is based on a predicted time for fluorescence to photobleach by 50% under arc lamp illumination with excitation intensity adjusted to produce 1000 emission photons per molecule per s.

FIG. 6 shows an example characterization of far-red FPs, consistent with various aspects of the present disclosure. The monomeric far-red FPs shown in FIG. 6 are mKate2 600, mNeptune1 605, mNeptune2 610, mNeptune2.5 615, mCardinal 620, and TagRFP657 625. For instance, FIG. 6A shows example mutation kinetics at 37° C. of the far-red FPs. For this experimental measurement, lysates from bacteria expressing FPs in anaerobic conditions were prepared beginning at time 0 and fluorescence measured over time. FIG. 6A shows the mean of triplicate measurements. FIG. 6B shows example green and red emissions detected of far-red FPs upon excitation with 460 nm light. As shown in FIG. 6B, far-red monomeric fluorescent proteins of the present disclosure have been found to demonstrate a reduced formation of a green fluorescent side-product that is common to eqFP578 derivatives and especially high in TagRFP657 as a proportion of total emission.

FIG. 6C shows example photobleaching kinetics of far-red FPs under arc lamp illumination with a 615/620 nm excitation filter. These experimental results are based on the mean times of three measurements that were normalized to spectral output of the lamp, transmission profiles of the filter and dichroic mirror, and FP absorbance spectra. As shown in FIG. 6C and Table 2, fluorescent protein variants, consistent with various experimental embodiments, show reduced photobleaching compared to mNeptune1. FIG. 6D shows example pH dependence of fluorescence of far-red FPs. As shown in FIG. 6D and Table 2, the fluorescence of mNeptune2.5 and mCardinal1 have been found to be more resistant to acidic conditions below pH 7. Further, the far-red fluorescent portions of the present disclosure have been found to retain the monomeric character of mNeptune1.

Various expression and detection conditions were researched to determine and demonstrate the utility of far-red fluorescent portions of the present disclosure in fluorescence microscopy of mammalian cells. For instance, when expressed in human embryonic kidney HEK293T cells and imaged with a 615/30 excitation filter, mNeptune2 and mCardinal1 were measured to be 100% and 30% brighter, respectively, than mNeptune1. This measured improvement was found to be greater than that in purified protein samples. This experimental result indicates better expression or maturation of mNeptune2 and mCardinal1 than mNeptune1 in mammalian cells. In contrast, mNeptune2.5 was 30% dimmer than mNeptune1 in HEK293T cells even though the purified protein was 40% brighter suggesting a defect in protein expression, chromophore maturation, protein stability, or chromophore stability. As mNeptune2.5 uniquely showed decreased fluorescence within minutes of reaching peak maturation in folding assays in vitro, FIG. 6A, it is postulated that the defect is in chromophore stability.

Experimental results demonstrate that mNeptune2 and mCardinal1 perform well in primary neurons as well as in primary mouse myoblasts in both proliferating and differentiated states. Fusion to mNeptune2 or mCardinal1 has been observed not to interfere with the subcellular localization of a wide variety of protein domains. Furthermore, mCardinal1 could be imaged with high contrast over background by laser-scanning confocal microscopy using a 633 nm laser, or by epifluorescence using a standard Cy5 filter set. Time-lapse imaging of mCardinal1 fusion proteins could be performed with a 633 nm laser at high magnification (60×) for over 60 frames without apparent toxicity.

To estimate and display the suitability for far-red fluorescent proteins of the present disclosure for imaging cells in mammalian tissues, brightness of mNeptune-related fluorescent proteins were compared to the blue-shifted relative mKate2 and TagRFP657 upon excitation by wavelengths in the optical window. When equal amounts of each purified protein were imaged in vitro using 625-655 nm excitation light, mCardinal1 was the brightest, followed by mNeptune2. mCardinal1 could also be detected with excitation by far-red light beyond 670 nm.

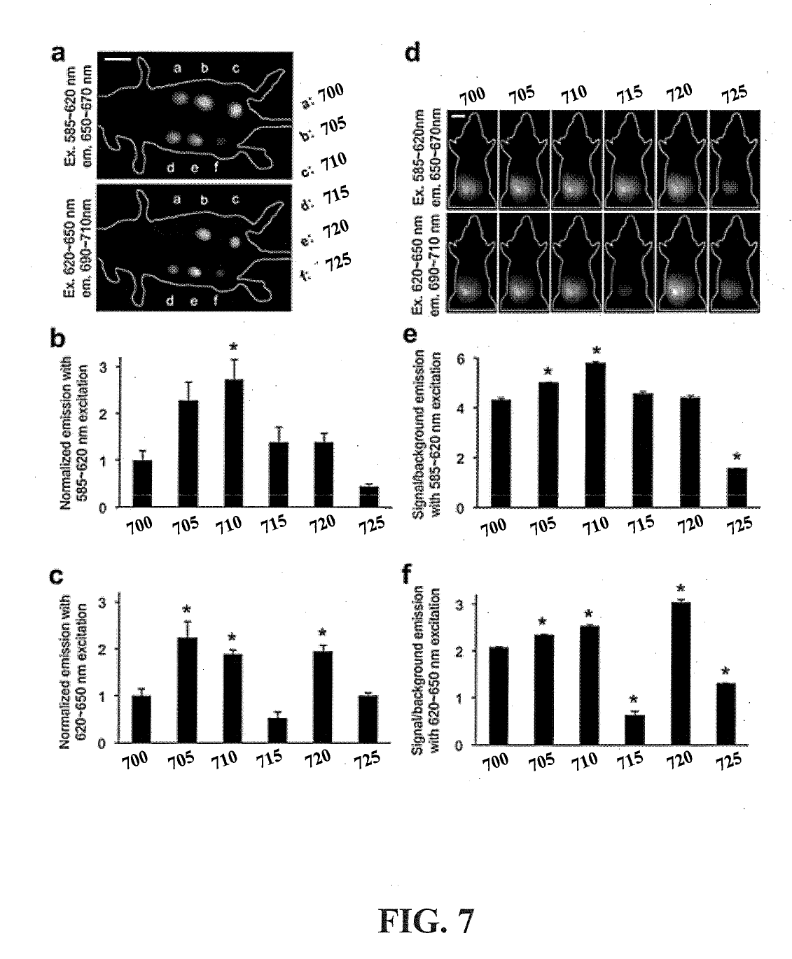

FIG. 7 shows comparison of far-red monomeric fluorescent proteins, consistent with various embodiments of the present disclosure, for deep-tissue imaging. The far-red FPs shown in FIG. 7 are mNeptune1 700, mNeptune2 705, mNeptune2.5 710, mKate2 715, mCardinal 720, and TagRFP657 725. FIG. 7A shows equal amounts (8 μg) of purified far-red FPs, consistent with various aspects of the present disclosure, injected subcutaneously into ventral locations in BALB/c nude mice. Each protein sample was imaged by epifluorescence with 585-620 nm or 620-650 nm excitation wavelengths (scale bar represents 1 cm). With 585-620 nm excitation light, a range that covers the excitation peaks of all the tested FPs, mNeptune2.5 was the brightest, which is consistent with this protein having the highest peak brightness in spectroscopic measurements, as demonstrated in Table 2. The experimental results of the 6 proteins injected are shown in FIG. 7B. FIG. 7B shows quantization of fluorescence of far-red FPs, consistent with various aspects of the present disclosure, relative to mNeptune1 upon excitation with a 585-620 nm filter. When using 620-650 nm light, however, mNeptune2, mNeptune2.5, and mCardinal1 were equally bright, as shown in FIG. 7C, consistent with the redder excitation spectra of mNeptune2 and mCardinal1 compared to mNeptune2.5, as shown in FIG. 4B. FIG. 7C shows example far-red FPs fluorescence, consistent with various aspects of the present disclosure, relative to mNeptune1 upon excitation with a 620-650 nm filter. The bars shown in FIGS. 7B and 7C are mean±standard error of the mean (SEM) of 5 replicates (differences are statistically significant by one-way ANOVA (p<0.0002). The asterisks indicate p<0.05 versus mNeptune1 by Tukey multiple comparison test.

To estimate the performance of far-red fluorescent proteins of the present disclosure in deeper tissue locations, a mouse phantom with optical transmission characteristics of mouse tissue that enabled precise placement of fluorescent protein samples were placed at a depth of 7 mm. FIG. 7D show an example measurement of equal amounts (23 μg) of purified far-red FPs placed inside of a phantom mouse in a bore located 7.2 mm deep from the mouse surface, consistent with various aspects of the present disclosure. Each protein sample was imaged by epifluorescence mode with 585-620 nm or 620-650 nm excitation wavelengths (scale bar represents 1 cm). Using 620-650 nm excitation light, mCardinal1 was uniquely brighter than all other far-red fluorescent proteins, as shown in FIG. 7F. FIG. 7E shows an example measurement of far-red FP contrast over the background (mean±SEM, n=3) upon excitation with a 585-620 nm filter, consistent with various aspects of the present disclosure. FIG. 7F shows an example measurement of far-red FP contrast over the background (mean±SEM, n=3) upon excitation with a 620-650 nm filter, consistent with various aspects of the present disclosure. This showing with respect to mCardinal1 at 7 mm depth is consistent with expected wavelength-dependent attenuation of excitation and emission light while traversing the phantom. Transmission of bluer photons in the 620-650 nm excitation band is attenuated more than the red photons by tissue, and this effect becomes more significant with depth. Likewise, mCardinal1's more red-shifted emission spectrum compared to mNeptune2 and mNeptune2.5 is expected to allow more photons to escape from tissue absorbance. Because mCardinal1 was found to become brighter relative to other proteins at redder excitations, it is expected to perform especially well in these deep imaging conditions. Taken together, these experimental results indicate that mCardinal1 performs well in deep-tissue imaging with wavelengths of light above 620 nm.

Far-red fluorescent monomeric proteins, consistent with the present disclosure, can be utilized for non-invasive optical imaging in terms of tracking cellular differentiation. This utility of the far-red fluorescent monomeric proteins, consistent with the present disclosure was experimentally tested by using mCardinal1 to visualize differentiation of transplanted myoblasts in skeletal muscle. Injury of the anterior tibialis (TA) muscle in a mouse was used as a model for muscle regeneration. One million myoblasts, stably expressing mCardinal1, were injected along the long axis of notexin-damaged TA muscle of SCID mice.

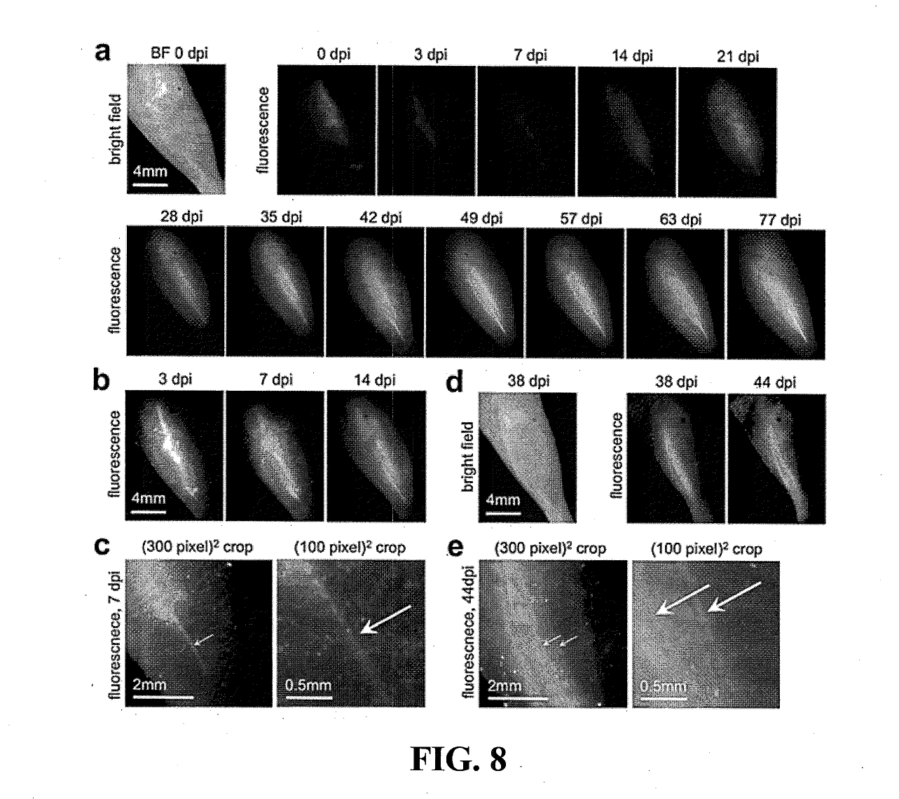

FIG. 8 shows non-invasive longitudinal visualization of muscle regeneration, consistent with various aspects of the present disclosure. FIG. 8A shows example representative fluorescence images of tibialis anterior muscles injected with 1 million myoblasts expressing mCardinal1, consistent with various aspects of the present disclosure. Fluorescence images were acquired using a fluorescence stereoscope with 615/30 nm excitation, and all images are normalized to the same intensity scale. FIG. 8B shows example acquired images from 3, 7, and 14 days post-injection (dpi), consistent with various aspects of the present disclosure. These images are shown having intensity scaling tighter than that used in (a) by a factor of 10, 10, and 5 respectively. FIG. 8C shows a magnified view of a muscle at 7 dpi, consistent with various aspects of the present disclosure. This shows an early regenerating fiber (arrow). The image at right is deliberately enlarged until pixelated to demonstrate that this regenerating fiber appears just a few pixels wide. The legs of these mice were imaged periodically, the experimental results of which are shown in FIGS. 8A-C. The injected cell clumps were visible by fluorescence immediately after injection and declined in the first 7 days post-injection (dpi). Beginning at 3 days post-injection, thin fluorescent fibers appeared in the leg, gradually increasing in number over the next 46 days and then reaching steady state. These results experimental demonstrate that fluorescence imaging of mCardinal1, and the other far-red fluorescent proteins of the present disclosure, can reveal tissue incorporation of newly differentiated cells in a mouse model of muscle regeneration non-invasively and with high spatial resolution.

Stem cell therapy is an actively researched method for repairing damaged or degenerated muscles. Stem cell performance varies by not only implantation timing and dosage, but by cell source and growth conditions ex vivo and in vivo. Stem cell research can benefit from a reliable method for visualizing muscle differentiation non-invasively and with high anatomical resolution. Therefore, the usability of the far-red fluorescent monomeric proteins of the present disclosure to track the fate of stem cells in living mice was investigated. In order to experimentally test the usability the far-red fluorescent monomeric proteins of the present disclosure, 1000 stems cells, cells stably transduced with a mCardinal1- and luciferase-expressing lentivirus, were injected into the TA muscles of SCID mice following damage by 18-Gy irradiation and notexin. After a second re-injury with notexin 30 days later, strong fluorescence signal was observed with the morphology of differentiated myofibers throughout a large region of the TA muscle, as shown in FIG. 8D. FIG. 8D shows an example fluorescence signal from tibialis anterior muscles injected with 1000 satellite cells expressing mCardinal1, consistent with various aspects of the present disclosure. FIG. 8E shows an example magnified view of muscle at 44 dpi injected with satellite cells, consist with various aspects of the present disclosure. The arrows indicate instances of multiple regenerating fibers. Contrast over background from a 1-min exposure was 11 (calculated as mean fluorescence of a region containing bright fibers divided by that of the equivalent region on a non-injected leg). As a result of the high contrast demonstrated by mCardinal1, fluorescence imaging of the far-red fluorescent monomeric proteins of the present disclosure can be used to visualize differentiation and tissue incorporation of stem cell derivatives longitudinally in situ in living animals.

Bioluminescence achieves higher detection sensitivity than fluorescence in whole animals due to negligible background. Therefore, the far-red fluorescent monomeric proteins of the present disclosure were experimentally tested to determine visualization of myofibers derived from stem cells using bioluminescence imaging of luciferase. Experimental results demonstrated an 8-min acquisition of bioluminescence signal at the same pixel sampling resolution showing a diffuse oval signal without any structural features, with a signal/noise ratio of 8.8. The inability to observe structural features in the bioluminescence signal may be related to the low light output of luciferase in two ways. First, despite essentially no dark current due to effective CCD cooling in the bioluminescence imaging device, the remaining noise sources of photonic shot noise and camera read noise combined to reach 10% of the brightest intensity values, resulting in discernible graininess of the image. Because myofibers may be as thin as the specimen area sampled by one pixel (26×26 μm), this graininess may have obscured detail. Second, micron-sized movements during the 8-minute acquisition such as muscle twitches could have blurred the image, especially if the leg did not return to the same precise position after movement. Thus, visualizing stem cell differentiation with sub-mm-level resolution may be difficult in practice with bioluminescence imaging.

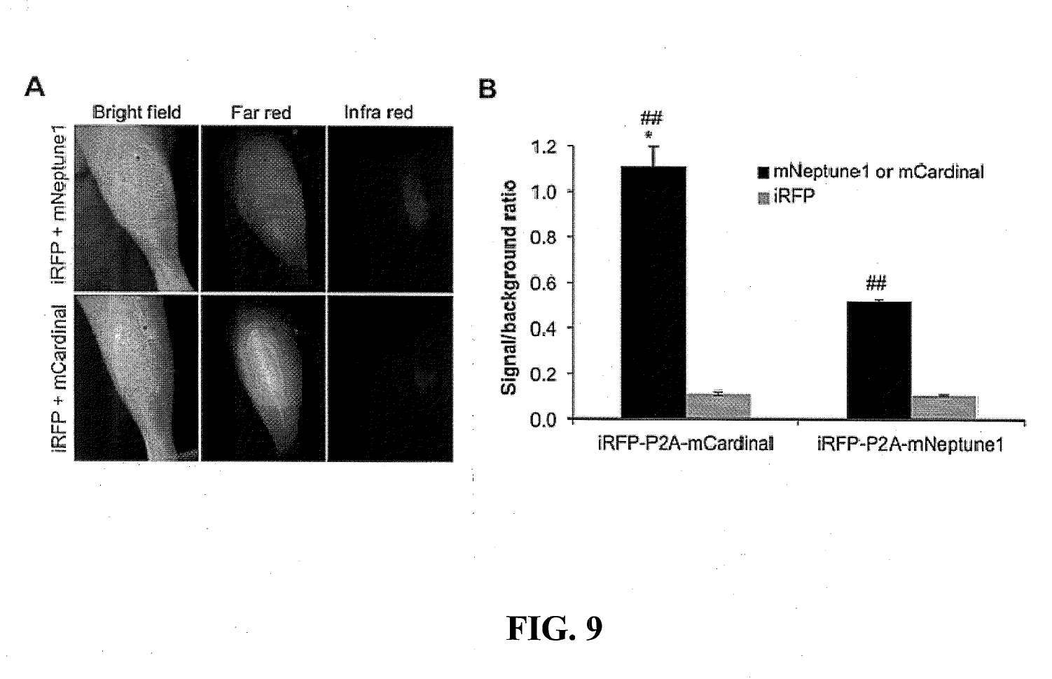

FIG. 9 shows an example comparison of mCardinal with mNeptune1 combined with iRFP in myoblasts, consistent with various aspects of the present disclosure. FIG. 9A shows representative fluorescence images of tibialis anterior (TA) muscles injected with 1 million myoblasts expressing mNeptune1 or mCardinal and iRFP separated with P2A peptide. Far-red and infrared fluorescence images (55 days post injection) were acquired a fluorescence stereoscope with 615/30 nm and 675/20 nm excitation, respectively. All fluorescence images are normalized to the same intensity scale. FIG. 9B shows an example contrast of mNeptune1 (combined with iRFP) versus mCardinal (combined with iRFP) over background.

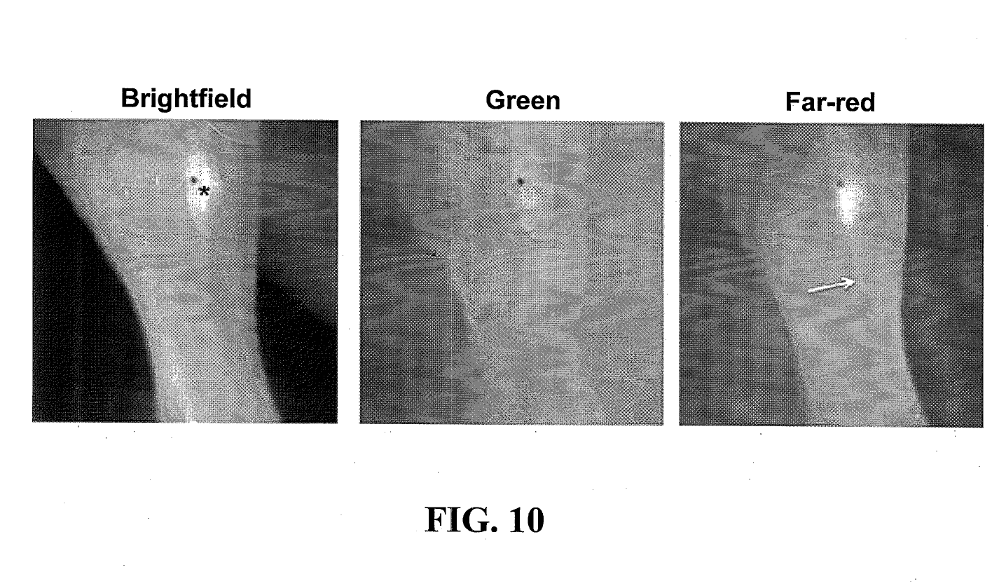

FIG. 10 shows an example comparison of mCardinal with the brightest GFP, Clover, in muscle stem cells, consistent with various aspects of the present disclosure. Representative fluorescence images of tibialis anterior (TA) muscles injected with 1000 stem cells expressing mCardinal and Clover separated with IRES sequence. Far-red and Clover fluorescence images (24 days post injection) were acquired a fluorescence stereoscope with 615/30 nm and 475/30 nm excitation, respectively. The asterisk denotes leg bone. Arrow indicates newly formed muscle fibers. Although Clover is the brightest fluorescent protein at any wavelength, its brightness is not sufficient to overcome autofluorescence from tissue. The absorption of green autofluorescence by blood is responsible for the outline of blood vessels.

During fluorescence imaging, photon fluxes are orders of magnitude higher than during bioluminescence imaging, and the limiting factor in detection is the ratio of specific signal to background autofluorescence. Tissue produces background fluorescence, and thus the concentration and molar brightness of a FP must be sufficiently high to create a signal over background. Once that condition is achieved, however, shorter integration times are possible with fluorescent proteins, consistent with the present disclosure, than with luciferase. Fluorescent proteins, consistent with the present disclosure, are capable of emitting millions of photons per second per molecule, orders of magnitude more than luciferase, having been measured at 2.7 photons per second at peak. In macroscopic applications, illumination intensity becomes the limiting factor in determining exposure time for fluorescence detection. Using light from an inexpensive 32-watt light-emitting diode (LED) source passing through a low-numerical aperture lens of a standard variable-magnification dissecting stereoscope, myocytes can be non-invasively imaged with acquisition times as low as 5 seconds. Short imaging times have utility in allowing repositioning of a subject expressing fluorescent proteins, consistent with the present disclosure, and multiple acquisitions in one session. This repositioning is not possible with high-resolution bioluminescence because luciferase signal will decay appreciably over the minutes-long time frame of a single exposure. Faster imaging also reduces the length of time under anesthesia for a subject. Additionally, the acquisition time of far-red monomeric fluorescence proteins, consistent with the present disclosure, can imaged in the sub-second range by utilizing, for example, higher-power light sources, higher-numerical aperture optics, or stereoscopes optimized for light capture and transmission.

Bioluminescence and fluorescence can have complementary roles in tracking cellular differentiation if a far-red monomeric fluorescent protein, consistent with various aspects of the present disclosure, and luciferase are coexpressed. The greater detection sensitivity of bioluminescence is useful for detecting low numbers of cells, discerning general trends in cell number and location, and screening multiple animals in wide fields of view. Non-invasive fluorescence imaging can be used to observe specific interesting regions at higher magnification. Cells can then be observed in their physiological contexts over time to assess how experimental conditions influence their survival or differentiation. Additionally, a far-red monomeric fluorescent protein, consistent with various aspects of the present disclosure, are desirable in microscopy when excitation with 633-635 nm lasers is needed, either for multi-wavelength imaging or to avoid autofluorescence from endogenous compounds. In particular, the far-red monomeric fluorescent proteins of the present disclosure improve fluorescence imaging of cells such as hepatocytes, neurons, or retinal pigment epithelial cells that contain high levels of lipofuscin, which is excited efficiently by all visible wavelengths below 620 nm. With its high brightness (compared to some fluorescent proteins excitable beyond 600 nm) and excitability with red light, far-red monomeric fluorescent proteins, consistent with various aspects of the present disclosure, are useful in a wide variety of live imaging applications. For further discussion of the various fluorescent monomeric proteins, as relating to the embodiments and specific applications discussed herein, reference may be made to the underlying provisional patent application (including the Appendices therein) to which priority is claimed. Reference may also be made to the published article to (and the supplementary information included in the provisional application) which is, together with the references cited therein, herein fully incorporated by reference. The aspects discussed therein may be implemented in connection with one or more of embodiments and implementations of the present disclosure (as well as with those shown in the figures). Moreover, for general information and for specifics regarding applications and implementations to which one or more embodiments of the present disclosure may be directed to and/or applicable, reference may be made to the references cited in the aforesaid patent application and published article, which are fully incorporated herein by reference generally and for the reasons noted above. In view of the description herein, those skilled in the art will recognize that many changes may be made thereto without departing from the spirit and scope of the present disclosure.

Based upon the above discussion and illustrations, those skilled in the art will readily recognize that various modifications and changes may be made without strictly following the exemplary embodiments and applications illustrated and described herein. Furthermore, various features of the different embodiments may be implemented in various combinations. Such modifications do not depart from the true spirit and scope of the present disclosure, including those set forth in the following claims.

SEQUENCES
mNeptune2-DNA sequence
ATGGTGTCTAAGGGCGAAGAGCTGATTAAGGAGAACATGCACATGAAGCTGTACAT
GGAGGGCACCGTGAACAACCACCACTTCAAGTGCACATCCGAGGGCGAAGGCAAGC
CCTACGAGGGCACCCAGACCGGCAGAATCAAGGTGGTCGAGGGCGGCCCTCTCCCC
TTCGCCTTCGACATCCTGGCTACCTGCTTCATGTACGGCAGCAAGACCTTCATCAAC
CACACCCAGGGCATCCCCGATTTCTTTAAGCAGTCCTTCCCTGAGGGCTTCACATGG
GAGAGAGTCACCACATACGAAGACGGGGGCGTGCTTACCGTTACCCAGGACACCAG
CCTCCAGGACGGCTGCTTGATCTACAACGTCAAGCTCAGAGGGGTGAACTTCCCATC
CAACGGCCCTGTGATGCAGAAGAAAACACTCGGCTGGGAGGCCAGTACCGAGACCC
TGTACCCCGCTGACGGCGGCCTGGAAGGCAGATGCGACATGGCCCTGAAGCTCGTG
GGCGGGGGCCACCTGCACTGCAACCTGAAGACCACATACAGATCCAAGAAACCCGC
TAAGAACCTCAAGATGCCCGGCGTCTACTTTGTGGACCGCAGACTGGAAAGAATCA
AGGAGGCCGACAATGAGACCTACGTCGAGCAGCACGAGGTGGCTGTGGCCAGATAC
TGCGACCTCCCTAGCAAACTGGGGCACAAACTTAATGGCATGGACGAGCTGTACAA
G
mNeptune2-protein sequence
MVSKGEELIKENMHMKLYMEGTVNNHHFKCTSEGEGKPYEGTQTGRIKVVEGGPLPFA
FDILATCFMYGSKTFINHTQGIPDFFKQSFPEGFTWERVTTYEDGGVLTVTQDTSLQDGC
LIYNVKLRGVNFPSNGPVMQKKTLGWEASTETLYPADGGLEGRCDMALKLVGGGHLH
CNLKTTYRSKKPAKNLKMPGVYFVDRRLERIKEADNETYVEQHEVAVARYCDLPSKLG
HKLNGMDELYK
mNeptune2.5-DNA sequence
ATGGTGAGCAAGGGCGAGGAGCTGATCAAGGAGAACATGCACACCAAGCTGTACAT
GGAAGGCACCGTGAACAACCACCACTTCAAGTGCACCCACGAAGGGGAGGGCAAG
CCCTACGAGGGCACCCAGACCAACAGGATTAAGGTGGTGGAGGGAGGCCCCCTGCC
GTTCGCATTCGACATCCTGGCCACCTGCTTTATGTACGGGAGCAAGACCTTCATCAA
CCACACCCAGGGCATCCCCGATTTCTTTAAGCAGTCCTTCCCTGAGGGCTTCACATG
GGAGAGAGTCACCACATACGAAGACGGGGGCGTGCTTACCGTTACCCAGGACACCA
GCCTCCAGGACGGCTGCTTGATCTACAACGTCAAGCTCAGAGGGGTGAACTTCCCAT
CCAACGGCCCTGTGATGCAGAAGAAAACACTCGGCTGGGAGGCCAGTACCGAGACC
CTGTACCCCGCTGACGGCGGCCTGGAAGGCAGATGCGACATGGCCCTGAAGCTCGT
GGGCGGGGGCCACCTGCACTGCAACCTGAAGACCACATACAGATCCAAGAAACCCG
CTAAGAACCTCAAGATGCCCGGCGTCTACTTTGTGGACCGCAGACTGGAAAGAATC
AAGGAGGCCGACAATGAGACCTACGTCGAGCAGCACGAGGTGGCTGTGGCCAGATA
CTGCGACCTCCCTAGCAAACTGGGGCACAAACTTAATGGCATGGACGAGCTGTACA
AG
mNeptune2.5-protein sequence
MVSKGEELIKENMHTKLYMEGTVNNHHFKCTHEGEGKPYEGTQTNRIKVVEGGPLPFA
FDILATCFMYGSKTFINHTQGIPDFFKQSFPEGFTWERVTTYEDGGVLTVTQDTSLQDGC
LIYNVKLRGVNFPSNGPVMQKKTLGWEASTETLYPADGGLEGRCDMALKLVGGGHLH
CNLKTTYRSKKPAKNLKMPGVYFVDRRLERIKEADNETYVEQHEVAVARYCDLPSKLG
HKLNGMDELYK
mCardinal1-DNA sequence
ATGGTGAGCAAGGGCGAGGAGCTGATCAAGGAGAACATGCACATGAAGCTGTACAT
GGAAGGCACCGTGAACAACCACCACTTCAAGTGCACCACCGAAGGGGAGGGCAAG
CCCTACGAGGGCACCCAGACCCAGAGGATTAAGGTGGTGGAGGGAGGCCCCCTGCC
GTTCGCATTCGACATCCTGGCCACCTGCTTTATGTACGGGAGCAAGACCTTCATCAA
CCACACCCAGGGCATCCCCGATTTCTTTAAGCAGTCCTTCCCTGAGGGCTTCACATG
GGAGAGAGTCACCACATACGAAGACGGGGGCGTGCTTACCGTTACCCAGGACACCA
GCCTCCAGGACGGCTGCTTGATCTACAACGTCAAGCTCAGAGGGGTGAACTTCCCAT
CCAACGGCCCTGTGATGCAGAAGAAAACACTCGGCTGGGAGGCCACCACCGAGACC
CTGTACCCCGCTGACGGCGGCCTGGAAGGCAGATGCGACATGGCCCTGAAGCTCGT
GGGCGGGGGCCACCTGCACTGCAACCTGAAGACCACATACAGATCCAAGAAACCCG
CTAAGAACCTCAAGATGCCCGGCGTCTACTTTGTGGACCGCAGACTGGAAAGAATC
AAGGAGGCCGACAATGAGACCTACGTCGAGCAGCACGAGGTGGCTGTGGCCAGATA
CTGCGACCTCCCTAGCAAACTGGGGCACAAACTTAATGGCATGGNCGAGCTGTACA
AG
mCardinal1-protein sequence
MVSKGEELIKENMHMKLYMEGTVNNHHFKCTTEGEGKPYEGTQTQRIKVVEGGPLPFA
FDILATCFMYGSKTFINHTQGIPDFFKQSFPEGFTWERVTTYEDGGVLTVTQDTSLQDGC
LIYNVKLRGVNFPSNGPVMQKKTLGWEATTETLYPADGGLEGRCDMALKLVGGGHLH
CNLKTTYRSKKPAKNLKMPGVYFVDRRLERIKEADNETYVEQHEVAVARYCDLPSKLG
HKLNGMXELYK
mCardinal2-DNA sequence
ATGGTGAGCAAGGGCGAGGAGCTGATCAAGGAGAACATGCCCATGAAGCTGTACAT
GGAAGGCACCGTGAACAACCACCACTTCAAGTGCACCACCGAAGGGGAGGGCAAG
CCCTACGAGGGCACCCAGACCCAGAGGATTAAGGTGGTGGAGGGAGGCCCCCTGCC
GTTCGCATTCGACATCCTGGCCACCTGCTTTATGTACGGGAGCAAGACCTTCATCAA
GCACCCCAAGGGCATCCCCGATTTCTTTAAGCAGTCCTTCCCTGAGGGCTTCACATG
GGAGAGAGTCACCACATACGAAGACGGGGGCGTGCTTACCGTTACCCAGGACACCA
GCCTCCAGGACGGCTGCTTGATCTACAACGTCAAGCTCAGAGGGGTGAACTTCCCAT
CCAACGGCCCTGTGATGCAGAAGAAAACACTCGGCTGGGAGGCCACCACCGAGACC
CTGTACCCCGCTGACGGCGGCCTGGAAGGCAGATGCGACATGGCCCTGAAGCTCGA
CGGCGGGGGCCACCTGCACTGCAACCTGAAGACCACATACAGATCCAAGAAACCCG
CTGGCAACCTCAAGATGCCCGGCGTCTACTTTGTGGACCGCAGACTGGAAAGAATC
AAGGAGGCCGACAATGAGACCTACGTCGAGCAGCACGAGGTGGCCGAGGCCAGAT
ACTGCGACCTCCCTAGCAAACTGGGGCACAAACTTAATGGCATGGACGAGCTGTAC
AAG
mCardinal2-protein sequence
MVSKGEELIKENMPMKLYMEGTVNNHHFKCTTEGEGKPYEGTQTQRIKVVEGGPLPFA
FDILATCFMYGSKTFIKHPKGIPDFFKQSFPEGFTWERVTTYEDGGVLTVTQDTSLQDGC
LIYNVKLRGVNFPSNGPVMQKKTLGWEATTETLYPADGGLEGRCDMALKLDGGGHLH
CNLKTTYRSKKPAGNLKMPGVYFVDRRLERIKEADNETYVEQHEVAEARYCDLPSKLG
HKLNGMDELYK

What is claimed is:

1. A fluorescent monomeric protein derived from Entacmeae quadricolor and configured and arranged with an excitation peak of above 590 nm and brightness at the excitation peak that is at least 20 mM⁻¹ cm⁻¹.

2. The protein of claim 1, wherein the fluorescent monomeric protein is a derivative of wild type eqFP578 protein from Entacmeae quadricolor.

3. The protein of claim 1, wherein the brightness of the fluorescent monomeric protein at 635 nm is at least 2 mM⁻¹ cm⁻¹.

4. The protein of claim 1, wherein the fluorescent monomeric protein is configured and arranged with a molar absorption coefficient, at 635 nm, that is at least 12 mM⁻¹ cm⁻¹.

5. The protein of claim 1, wherein the fluorescent monomeric protein includes a mutation to at least one position in a beta barrel wall of the fluorescent monomeric protein.

6. The protein of claim 1, wherein the fluorescent monomeric protein includes a mutation to at least one position in a hydrophobic core of the fluorescent monomeric protein.

7. The protein of claim 1, wherein the fluorescent monomeric protein includes a sequence of one of mNeptune2, mNeptune2.5, mCardinal1, mCardinal2.

8. The protein of claim 1, wherein the fluorescent monomeric protein includes a point mutations to mNeptune1 at one or more of locations S28, G41, M11, S143 and providing an excitation peak that is above 590 nm.

9. The protein of claim 1, wherein the fluorescent monomeric protein includes point mutations to mNeptune1 at location S28 and G41 and providing an excitation peak that is above 590 nm.

10. The protein of claim 1, wherein the fluorescent monomeric protein includes point mutations to mNeptune1 at location S28, G41 and M11 and providing an excitation peak that is above 590 nm.

11. The protein of claim 1, wherein the fluorescent monomeric protein includes point mutations to mNeptune1 at location S28, G41 and S143 and providing an excitation peak that is above 590 nm.

12. A method comprising
providing a fluorescent monomeric protein derived from Entacmeae quadricolor and configured and arranged with an excitation peak of above 590 nm and brightness at the excitation peak that is at least 20 mM⁻¹ cm⁻¹ to a cell in vivo; and
using an excitation light of at least 600 nm to active the fluorescent monomeric protein.

13. The method of claim 12 wherein using the excitation light includes using at least 620 nm light.

14. The method of claim 12, further comprising co-expressing the fluorescent monomeric protein with a bioluminescence marker.

15. The method of claim 12, further including using an acquisition time of 5-8 seconds.


SEQUENCE LISTING (8 sequences)

SEQ ID NO: 1 - DNA, 732 bp, Entacmaea quadricolor
atggtgtctaagggcgaagagctgattaaggagaacatgcacatgaagctgtacatggagggcaccgtgaacaaccaccacttcaagtgcacatccgagggcgaaggcaagccctacgagggcacccagaccggcagaatcaaggtggtcgagggcggccctctccccttcgccttcgacatcctggctacctgcttcatgtacggcagcaagaccttcatcaaccacacccagggcatccccgatttctttaagcagtccttccctgagggcttcacatgggagagagtcaccacatacgaagacgggggcgtgcttaccgttacccaggacaccagcctccaggacggctgcttgatctacaacgtcaagctcagaggggtgaacttcccatccaacggccctgtgatgcagaagaaaacactcggctgggaggccagtaccgagaccctgtaccccgctgacggcggcctggaaggcagatgcgacatggccctgaagctcgtgggcgggggccacctgcactgcaacctgaagaccacatacagatccaagaaacccgctaagaacctcaagatgcccggcgtctactttgtggaccgcagactggaaagaatcaaggaggccgacaatgagacctacgtcgagcagcacgaggtggctgtggccagatactgcgacctccctagcaaactggggcacaaacttaatggcatggacgagctgtacaag

SEQ ID NO: 2 - protein, 244 aa, Entacmaea quadricolor [sequence omitted]

SEQ ID NO: 3 - DNA, 732 bp, Entacmaea quadricolor
atggtgagcaagggcgaggagctgatcaaggagaacatgcacaccaagctgtacatggaaggcaccgtgaacaaccaccacttcaagtgcacccacgaaggggagggcaagccctacgagggcacccagaccaacaggattaaggtggtggagggaggccccctgccgttcgcattcgacatcctggccacctgctttatgtacgggagcaagaccttcatcaaccacacccagggcatccccgatttctttaagcagtccttccctgagggcttcacatgggagagagtcaccacatacgaagacgggggcgtgcttaccgttacccaggacaccagcctccaggacggctgcttgatctacaacgtcaagctcagaggggtgaacttcccatccaacggccctgtgatgcagaagaaaacactcggctgggaggccagtaccgagaccctgtaccccgctgacggcggcctggaaggcagatgcgacatggccctgaagctcgtgggcgggggccacctgcactgcaacctgaagaccacatacagatccaagaaacccgctaagaacctcaagatgcccggcgtctactttgtggaccgcagactggaaagaatcaaggaggccgacaatgagacctacgtcgagcagcacgaggtggctgtggccagatactgcgacctccctagcaaactggggcacaaacttaatggcatggacgagctgtacaag

SEQ ID NO: 4 - protein, 244 aa, Entacmaea quadricolor
MVSKGEELIKENMHTKLYMEGTVNNHHFKCTHEGEGKPYEGTQTNRIKVVEGGPLPFAFDILATCFMYGSKTFINHTQGIPDFFKQSFPEGFTWERVTTYEDGGVLTVTQDTSLQDGCLIYNVKLRGVNFPSNGPVMQKKTLGWEASTETLYPADGGLEGRCDMALKLVGGGHLHCNLKTTYRSKKPAKNLKMPGVYFVDRRLERIKEADNETYVEQHEVAVARYCDLPSKLGHKLNGMDELYK

SEQ ID NO: 5 - DNA, 732 bp, Entacmaea quadricolor
  misc_feature (719)..(719): n is a, c, g, or t
atggtgagcaagggcgaggagctgatcaaggagaacatgcacatgaagctgtacatggaaggcaccgtgaacaaccaccacttcaagtgcaccaccgaaggggagggcaagccctacgagggcacccagacccagaggattaaggtggtggagggaggccccctgccgttcgcattcgacatcctggccacctgctttatgtacgggagcaagaccttcatcaaccacacccagggcatccccgatttctttaagcagtccttccctgagggcttcacatgggagagagtcaccacatacgaagacgggggcgtgcttaccgttacccaggacaccagcctccaggacggctgcttgatctacaacgtcaagctcagaggggtgaacttcccatccaacggccctgtgatgcagaagaaaacactcggctgggaggccaccaccgagaccctgtaccccgctgacggcggcctggaaggcagatgcgacatggccctgaagctcgtgggcgggggccacctgcactgcaacctgaagaccacatacagatccaagaaacccgctaagaacctcaagatgcccggcgtctactttgtggaccgcagactggaaagaatcaaggaggccgacaatgagacctacgtcgagcagcacgaggtggctgtggccagatactgcgacctccctagcaaactggggcacaaacttaatggcatggncgagctgtacaag

SEQ ID NO: 6 - protein, 244 aa, Entacmaea quadricolor
  misc_feature (240)..(240): Xaa can be any naturally occurring amino acid
MVSKGEELIKENMHMKLYMEGTVNNHHFKCTTEGEGKPYEGTQTQRIKVVEGGPLPFAFDILATCFMYGSKTFINHTQGIPDFFKQSFPEGFTWERVTTYEDGGVLTVTQDTSLQDGCLIYNVKLRGVNFPSNGPVMQKKTLGWEATTETLYPADGGLEGRCDMALKLVGGGHLHCNLKTTYRSKKPAKNLKMPGVYFVDRRLERIKEADNETYVEQHEVAVARYCDLPSKLGHKLNGMXELYK

SEQ ID NO: 7 - DNA, 732 bp, Entacmaea quadricolor
atggtgagcaagggcgaggagctgatcaaggagaacatgcccatgaagctgtacatggaaggcaccgtgaacaaccaccacttcaagtgcaccaccgaaggggagggcaagccctacgagggcacccagacccagaggattaaggtggtggagggaggccccctgccgttcgcattcgacatcctggccacctgctttatgtacgggagcaagaccttcatcaagcaccccaagggcatccccgatttctttaagcagtccttccctgagggcttcacatgggagagagtcaccacatacgaagacgggggcgtgcttaccgttacccaggacaccagcctccaggacggctgcttgatctacaacgtcaagctcagaggggtgaacttcccatccaacggccctgtgatgcagaagaaaacactcggctgggaggccaccaccgagaccctgtaccccgctgacggcggcctggaaggcagatgcgacatggccctgaagctcgacggcgggggccacctgcactgcaacctgaagaccacatacagatccaagaaacccgctggcaacctcaagatgcccggcgtctactttgtggaccgcagactggaaagaatcaaggaggccgacaatgagacctacgtcgagcagcacgaggtggccgaggccagatactgcgacctccctagcaaactggggcacaaacttaatggcatggacgagctgtacaag

SEQ ID NO: 8 - protein, 244 aa, Entacmaea quadricolor
MVSKGEELIKENMPMKLYMEGTVNNHHFKCTTEGEGKPYEGTQTQRIKVVEGGPLPFAFDILATCFMYGSKTFIKHPKGIPDFFKQSFPEGFTWERVTTYEDGGVLTVTQDTSLQDGCLIYNVKLRGVNFPSNGPVMQKKTLGWEATTETLYPADGGLEGRCDMALKLDGGGHLHCNLKTTYRSKKPAGNLKMPGVYFVDRRLERIKEADNETYVEQHEVAEARYCDLPSKLGHKLNGMDELYK